(19) United States
(12) Patent Application Publication
(10) Pub. No.: US 2006/0147565 A1
(43) Pub. Date: Jul. 6, 2006

(54) Purified amylase inhibitor and novel process for obtaining the same

(75) Inventors: Mitchell Skop, Closter, NJ (US); Dilip Chokshi, Parsippany, NJ (US)

Correspondence Address: Ronald J Baron;Hoffmann & Baron, 6900 Jericho Turnpike, Syosset, NY, 11791, US

(73) Assignee: PHARMACHEM LABORATORIES, INC., Kearny, NJ (US)

(21) Appl. No.: 10/539,098

(22) PCT Filed: Jun. 27, 2003
(86) PCT No.: PCT/US03/20459

§ 371 Date: Jun. 15, 2005

Related U.S. Application Data
(60) Provisional application No. 60/392,676, filed on Jun. 28, 2002.

(51) Int. Cl. A61K 36/48; C12N 9/99

(52) U.S. Cl. 424/757; 435/184

(57) ABSTRACT

The present invention provides a purified amylase inhibitor obtained by a superior process that includes extracting ground beans with supercritical carbon dioxide. The invention also provides a method for inducing weight loss in a mammal in need thereof comprising administering to the mammal, an effective amount of an amylase inhibitor obtained by the superior process. A method for improving post-prandial glucose tolerance in a diabetic mammal comprising administering to the mammal, an effective amount of an amylase inhibitor obtained by the superior process, is also provided.

### BACKGROUND OF THE INVENTION

Amylase is an enzyme responsible for breaking down the main source of carbohydrates in the human diet, namely, starch. The digestion of starch begins in the mouth where alpha-amylase present in saliva hydrolyzes glucosidic bonds of starch.

By the time thoroughly chewed food reaches the stomach, the average chain length of starch is reduced from several thousand to less than eight glucose units. The acid level in the stomach inactivates the salivary alpha-amylase. Further digestion of starch continues in the small intestine by pancreatic alpha-amylase, which is similar to that of salivary alpha-amylase.

Decreasing the absorption of carbohydrates by inhibiting the digestion of starch is a very promising strategy in the fields of, for example, weight loss and diabetes mellitus. From a dietary standpoint, it is important to target the breakdown of starch since starch is a relatively nonessential nutrient, which provides calories with little benefit.

Amylase inhibitors are derived from various sources, including vegetable albumins and leguminous plants. Currently, extracts from beans, are being utilized most often as a source of amylase inhibitors.

Current methods for purification of amylase inhibitors, which includes concentrating and drying beans, include the use of heat treatments and/or solvents. See U.S. Pat. No. 6,340,699 to Cestaro, et al. However, the use of heat treatments and/or solvents has several drawbacks. For example, at high temperatures, certain heat sensitive components of the amylase inhibitor from beans can become degraded. As a result, the amylase inhibitor exhibits a decrease in stability and potency.

In addition, there are environmental and health concerns associated with the use of solvents during such purification processes. For example, extraction of amylase inhibitors from beans using solvents results in residual contamination of the extract with the toxic solvent. Furthermore, disposal of the large quantities of solvent required during purification processes is a major environmental concern.

Amylase inhibitors that are derived from bean extracts by the conventional heat and solvent methods are not purified, i.e. they contain impurities and/or contaminants. Examples of such impurities are solvent residue and inactive components of the beans.

Amylase inhibitors are often added to food products for consumption, such as, for example, powdered drink mixes, prepared shakes, snack bars, etc. The impurities and/or contaminants that remain in the bean extract are associated with negative flavors that render such food products unappetizing.

Recent studies have indicated that the currently available amylase-inhibitors work well in vitro, but fail to be effective in vivo. Some of the proffered reasons are that the currently available amylase inhibitors are unstable in the gastrointestinal tract due to pH, are insoluble in water, and/or lose potency due to the use of solvents and heat treatments. See Layer, P. et al. Gastroenterology 1985; 88(6): 1895-1902.

Therefore, in light of the above deficiencies that exist with current amylase inhibitors, there is a need for a more pure and potent amylase inhibitor derived from beans, and a more sophisticated process for obtaining the same.

### SUMMARY OF THE INVENTION

These and other objectives have been met by the present invention by providing an amylase inhibitor obtained by a superior process. The process comprises grinding white kidney beans then extracting the ground beans under vacuum pressure with supercritical carbon dioxide to remove impurities, leaving a bean mass. The bean mass is then incubated with deionized water to obtain a first bean suspension that contains a first solid component and a first liquid component. The first solid component is separated out of the first bean suspension, while retaining the first liquid component. The first solid component is then incubated with deionized water to obtain a second bean suspension that contains a second solid component and a second liquid component. The second solid component is separated out of the second bean suspension, while retaining the second liquid component. The first and second liquid components are then combined to obtain a final liquid solution. The final liquid solution is subjected to heat exchange to obtain a concentrated bean extract. The concentrated bean extract is dried and a purified amylase inhibitor is obtained.

The invention also provides a method for inducing weight loss in a mammal in need thereof comprising administering to the mammal, an effective amount of an amylase inhibitor obtained by the superior process.

A method for improving post-prandial glucose tolerance in a diabetic mammal comprising administering to the mammal, an effective amount of an amylase inhibitor obtained by the superior process, is also provided.

### DETAILED DESCRIPTION OF THE INVENTION

Applicants have surprisingly discovered that a purified amylase inhibitor is obtainable by the novel process of the invention.

Amylase inhibitors are glycoproteins that inhibit the enzyme responsible for breaking down carbohydrates, namely, amylase. The amylase inhibitor of the invention is derived from beans. Suitable beans for use in the invention belong to the Phaseolus vulgaris family which includes, for example, kidney beans. Preferably, the amylase inhibitor is derived from white kidney beans. The amylase inhibitor from white kidney beans is sometimes referred to as “phaseolamin.” Preferably, the beans are not genetically modified beans. The beans are typically small, intact beans.

The amylase inhibitor of the invention is superior to other amylase inhibitors because it is of a higher degree of purity than amylase inhibitors obtained by conventional extraction methods, i.e. heat and chemical. Due to the high degree of purity, the amylase inhibitor exhibits improved stability and potency in vitro and in vivo over amylase inhibitors of the prior art.

The amylase inhibitor of the present invention remains stable at elevated temperatures, such as, for example, 120-200° F. Such heat stability allows the amylase inhibitor to be utilized in, for example, food products that are cooked, without losing the beneficial, starch-blocking effects.

The amylase inhibitor also remains intact at extreme pH values. For example, the stomach can have a pH of approximately 1-2. The amylase inhibitor of the invention remains mainly intact under such pH conditions.

In addition, the amylase inhibitor is more potent than the amylase inhibitors derived from conventional heat/solvent methods. Not being bound by theory, it is proposed that by avoiding the use of chemical solvents, the important tertiary structure of the amylase inhibitor is not disrupted.

According to the invention, white kidney beans are subject to grinding to produce coarsely ground beans. The beans are ground by any method known to those in the art. For example, the beans may be ground by manual or mechanical means. An example of a manual method for grinding includes a mortar and pestle. An example of a mechanical method for grinding includes a grinding mill, such as a Fitzpatrick Mill manufactured by Robinson.

The coarsely ground beans are subject to extraction to remove impurities as will be discussed below. The extraction step involves the use of supercritical carbon dioxide. Carbon dioxide exists under normal conditions, i.e. ambient temperature and pressure, as a gas. The critical temperature (Tc) for CO₂ is 31.06° C. (88° F.) and the critical pressure (Pc) is 73.8 bar. CO₂ is in a supercritical state when both the temperature and pressure is higher than its Tc and Pc. In a supercritical state, the CO₂ is essentially a compressed, high density fluid.

The coarsely ground beans are placed into an extraction vessel (i.e., extractor) and extracted with supercritical CO₂ under vacuum pressure. Vacuum pressure is typically any pressure which is below atmospheric pressure. In one embodiment, extraction occurs at a temperature of about 120° F. to 200° F. for about two hours. Preferably, the extraction step is performed at a temperature of about 135° F. to 160° F. for about two hours. More preferably, the extraction step is performed at a temperature of about 145° F. for about two hours.

During the extraction step, the supercritical CO₂ fluid passes through the ground beans and dissolves and extracts the impurities from the beans to form a supercritical solution. Thus, the supercritical solution contains impurities from the bean. The impurities are typically non-polar constituents of the beans and include, for example, lipids, oils, fats, and flavors.

After the extraction has been completed, the supercritical solution is removed from the extractor via a pressure reduction value. The pressure and the dissolving power of the supercritical fluid is reduced, thereby causing the impurities of the bean to precipitate in a separator. For purposes of this invention, the remaining product, substantially free of impurities, is referred to as a bean mass. The bean mass contains the glycoproteins, i.e. amylase inhibitors.

The bean mass is then incubated in deionized water to form a first bean suspension. Deionized water is typically water in which ions have been removed. The temperature of the deionized water is preferably from about 120° F. to about 160° F., more preferably, the deionized water is about 140° F. The bean mass is incubated in the deionized water for up to about 6 hours, more preferably for about 4 hours. During incubation, glycoproteins are extracted from the bean mass.

As mentioned above, after incubation, a first bean suspension is obtained. The first bean suspension contains a first solid component and a first liquid component. The first liquid component contains deionized water and glycoproteins (i.e. amylase inhibitor) from the bean mass. The first solid component contains any remaining unextracted components which includes, for example, impurities. The first solid component is then separated from the first liquid component. The first liquid component is retained in a separate container.

Separation is done by any means known in the art. For example, separation is accomplished by centrifugation or filtration. Filtration by filter press is preferred. For example, in filtration the first bean suspension is poured over a porous material (e.g., filter), such as a filter paper. The filter allows the passage of the liquid component through the filter and prevents passage of the solid component.

Centrifugation uses centrifugal force to promote solid and liquid separation. For example, in centrifugation the first bean suspension is placed in a tube. The tube in then placed in a centrifuge and centrifugal force is applied. As a result, the solid component gathers in the bottom portion of the tube (i.e. pellet), while the liquid component remains at the top (i.e. supernatant) portion of the tube. The liquid component is then poured off and retained in a separate container.

Once the first solid component is separated from the first liquid component, the first solid component is incubated, as described above, in deionized water to form a second bean suspension. The second bean suspension contains a second liquid component and a second solid component. The second liquid component contains deionized water and glycoproteins (i.e. amylase inhibitor) from the first solid component. The second solid component contains any remaining unextracted components which includes, for example, impurities. The second solid component is then separated from the second liquid component by any suitable means, including those discussed above. The second liquid component is retained in a separate container.

The first liquid component and second liquid component are then combined to obtain a final liquid solution. The final liquid solution is then subjected to heat exchange. Heat exchange is a distillation process, which removes water. Heat exchange preferably occurs under vacuum pressure. Apparatus suitable for heat exchange are known in the art.

As a result of the heat exchange step, water is removed from the final liquid solution to obtain a concentrated bean extract. The concentrated bean extract may contain approximately 25-50% water. More preferably, the concentrated bean extract contains approximately 35% water.

The concentrated bean extract is then dried. Drying of the final bean concentrate can be accomplished by any suitable means known in the art. For example, in one embodiment, the drying step is performed by lyophilizaton, i.e. freeze drying. The process of freeze drying removes residual water from the concentrated bean extract by sublimation and desorbtion.

During lyophilization, the concentrated bean extract is transported in a chilled vessel to a freeze dryer for drying. A condenser in the drying chamber of the freeze dryer traps water removed from the concentrated bean extract, while a vacuum system reduces pressure to facilitate the drying process. Once the lyophilization process is complete, a purified amylase inhibitor is obtained.

In another embodiment, the drying step is performed by utilizing a spray dryer. The spray dryer consists of a feed pump, atomizer, air heater, air dispenser, drying chamber, and systems for exhaust air cleaning and powder recovery. Air or gas can be used in spray drying. An example of a hot gas which can be used in a spray dryer, includes, but is not limited to, nitrogen.

The nozzle used in the spray drying process can be, for example, a centrifugal wheel nozzle or a high pressure nozzle. The input (inlet) temperature of the hot air used for spray drying is from about 400° F. to about 500° F., and preferably about 440° F. The output (outlet) temperature of the hot air used for spray drying is from about 150° F. to about 250° F., and preferably about 210° F. In the spray drying process, the concentrated bean extract is sprayed into hot gas, thereby converting the concentrated bean extract into a free flowing particulate dried bean extract.

After spraying drying, the dried bean extract is rehydrated to obtain a rehydrated bean extract. Rehydration is accomplished by the addition of water to the dried bean extract. Preferably, the water is deionized. In a preferred embodiment, approximately 40-70% of the dried bean extract is rehydrated. More preferably approximately 60% of the dried bean extract is rehydrated.

The rehydrated bean extract is then lyophilized, i.e. freeze dried, as described above. The rehydrated bean extract is subjected to freeze drying to obtain the purified amylase inhibitor.

The primary function of amylase inhibitors is to cause temporary, safe, side-effect free malabsorption of dietary starch. Not being bound by theory, it is believed that the amylase inhibitor of the invention binds to, and neutralizes, alpha-amylase. By neutralizing alpha-amylase, absorption of carbohydrates is inhibited. As will be discussed below, the amylase inhibitor is effective for inducing weight loss.

As discussed above, alpha-amylase is a naturally occurring starch enzyme that is responsible for the breakdown of starches. For example, in humans, dietary starches must be broken down into smaller components, such as glucose, in order to be utilized by the body. Starches that are consumed, but are not broken down into smaller components, such as glucose, are not utilized in vivo. Therefore, by neutralizing the body's alpha amylase, the body's ability to use starches is hindered, and ultimately the unused starches are excreted.

Digestion, or the breakdown of starch into glucose, triggers the production of insulin. Hence, consuming a starch-rich meal causes an abnormal rise in insulin. Excess insulin triggers hunger and cravings, creating a vicious cycle. One way to end the cycle is to reduce or eliminate the intake of starches. This approach has had very little or no success in inducing weight loss for the long term.

In one embodiment of the invention, a method for inducing weight loss in a mammal in need thereof is provided. The method comprises administering to the mammal an effective amount of the amylase inhibitor of the invention.

A mammal in need of weight loss is, for example, any mammal whose weight is detrimental to its health. Another example of a mammal in need of weight loss is, for example, a mammal that is unhappy with its appearance due to excess weight. Excess weight of a mammal is subjective. Some examples of mammals in need of weight loss include, but are not limited to, mammals that suffer from diabetes mellitus and/or obesity.

Not being bound by theory, it is believed that the highly pure amylase inhibitor of the invention induces weight loss by inhibiting the absorption of starches. In addition, the amylase inhibitor controls cravings associated with carbohydrate absorption. By inhibiting absorption of dietary starch and controlling cravings associated with carbohydrate absorption, the amylase inhibitor is effective in inducing weight loss.

The amylase inhibitor of the claimed invention is also used in a mammal suffering from an impairment of glucose utilization, for example, diabetes mellitus. The impairment in glucose utilization may occur as a result of a deficiency in the production of insulin by the pancreas, or by ineffectiveness of the insulin produced to utilize glucose. As will be discussed below, insulin is necessary to the transport of glucose from the blood into cells.

Insulin is a hormone naturally produced by the body that is key to controlling blood glucose levels. Circulating blood carries glucose that provides fuel for the cells. Getting glucose into the cells requires insulin, which is produced in the pancreas by beta cells. Normally, the pancreas produces just enough insulin to handle the body's needs. This is not the case with hyperglycemia disorders, such as diabetes mellitus (DM), as will be discussed below.

In DM, insulin is either absent, in short supply or unable to perform its job efficiently. If glucose cannot get into the cells, it accumulates in the blood creating increased blood glucose. The amount of glucose in the blood after consumption of a meal is the postprandial glucose level.

For example, in people who do not have DM, the plasma glucose levels peaks about one hour after a meal and returns to pre-meal levels within two to three hours after a meal. In contrast, those that suffer from DM, the postprandial glucose increases to a higher level and lasts longer compared to those individuals without diabetes. An impairment in postprandial glucose tolerance can lead to the development of, for example, cardiovascular disease.

Individuals suffering from DM usually need to ingest insulin to aid in the absorption of blood glucose into cells. Often, after consuming a carbohydrate rich meal, a diabetic's insulin requirements may markedly increase to deal with the high blood glucose levels.

Accordingly, by inhibiting the absorption of dietary starch, the amylase inhibitor of the present invention will effectively decrease the insulin requirements of a diabetic mammal. In addition, the amylase inhibitor of the present invention will also lower the level of postprandial glucose in the blood, thereby improving postprandial glucose tolerance. Hence, in another embodiment of the invention, a method for improving postprandial glucose tolerance in a diabetic mammal is provided.

Preferably, the amylase inhibitor is administered systemically. Systemic administration can be enteral or parenteral. Enteral administration is preferred. For example, the amylase inhibitor is easily administered orally. Liquid or solid (e.g., tablets, gelatin capsules) formulations can be employed. The formulations can include pharmaceutically acceptable excipients, adjuvants, diluents, or carriers.

The amylase inhibitor is also administered in chewable tablet granulations, with or without sugar, in powdered drink mixes, chewing gum and baking products. In a preferred embodiment, because the amylase inhibitor is stable under baking temperatures, it is effectively administered in baking mixes such as pancakes, waffles, breads, biscuits or cookies.

In accordance with the present invention, an effective amount of the amylase inhibitor is any amount known to those skilled in the art to effectively inhibit the breakdown of dietary starch. Preferably, an effective amount is administered to a mammal just prior to, during, or shortly after, consuming a starch-rich meal. For example, a typical pre-meal dosage of the amylase inhibitor is approximately 500 mg to 1,500 mg.

In accordance with the invention, mammals include, for example, humans, as well as pet animals such as dogs and cats, laboratory animals such as rats and mice, and farm animals such as horses and cows. Humans are most preferred.

### EXAMPLE 1

### Purification of Amylase Inhibitor by Spray Drying

1. Grinding

Whole, dried, non-genetically modified organism (non-GMO) Phaseolus vulgaris beans were inspected for cleanliness. Upon quality control approval of the beans, 1000 g of the dried beans were placed into a Fitzpatrick® grinding mill. A #4 screen (course ground size) was used in the grinding mill. The grinding continued until the beans were the appropriate size.

2. Extraction

The coarsely ground beans were placed into an extraction vessel and extracted with supercritical CO₂. The extraction process occurs under vacuum pressure at about 145° F. for about two hours. The supercritical CO2 removes the impurities (e.g., lipids, oils, fats, and flavors, etc.) from the coarsely ground beans, leaving a bean mass.

The pressure in the extraction vessel was then reduced. The reduction causes the impurities from the bean to precipitate from the supercritical solution and into a separator.

3. Incubation

Deionized water at 140° F. was added to the bean mass and allowed to incubate for 4 hours. During incubation, glycoproteins were extracted from the bean mass. A first bean suspension was obtained.

4. Separation

The first bean suspension was then filtered by a filter press to separate the solid components from the bean suspension. The first liquid component (containing glycoproteins) was retained in a separate container. The first solid component was then incubated in deionized water as above to form a second bean suspension. The second bean suspension was then filtered as above to separate the second solid components from the second liquid components. The first and second liquid components were then combined to obtain a final liquid solution.

5. Heat Exchange

The final liquid solution was subjected to heat exchange to remove water and to obtain a concentrated bean extract.

6. Drying

The concentrated bean extract was then subjected to spray drying to remove the residual water. A high pressure nozzle was used for the spray drying procedure. The concentrated bean extract was subjected to hot air with an input temperature of 440° F. and an output temperature of 210° F., until a dried bean extract was formed.

Approximately 40% (w/w) of the dried bean extract was rehydrated with deionized water. The rehydrated bean extract was then lyophilized to obtain the purified amylase inhibitor. From the 1000 g of dried beans, approximately 120 g of purified amylase inhibitor was obtained.

### EXAMPLE 2

### Purification of Amylase Inhibitor by Drying With Lyophilization

1. Grinding

Whole, dried, non-genetically modified organism (non-GMO) Phaseolus vulgaris beans were inspected for cleanliness. Upon quality control approval of the beans, 1000 g of the dried beans were placed into a Fitzpatrick® grinding mill. A #4 screen (course ground size) was used in the grinding mill. The grinding continued until the beans were the appropriate size.

2. Extraction

The coarsely ground beans were placed into an extraction vessel and extracted with supercritical CO₂. The extraction process occurs under vacuum pressure at about 14520 F. for about two hours. The supercritical CO2 removes the impurities (e.g., lipids, oils, fats, and flavors, etc.) from the coarsely ground beans, leaving a bean mass.

The pressure in the extraction vessel was then reduced. The reduction causes the impurities from the bean to precipitate from the supercritical solution and into a separator.

3. Incubation

Deionized water at 140° F. was added to the bean mass and allowed to incubate for 4 hours. During incubation, glycoproteins were extracted from the bean mass. A first bean suspension was obtained.

4. Separation

The first bean suspension was then filtered by a filter press to separate the solid components from the bean suspension. The first liquid component (containing glycoproteins) was retained in a separate container. The first solid component was then incubated in deionized water as above to form a second bean suspension. The second bean suspension was then filtered as above to separate the second solid components from the second liquid components. The first and second liquid components were then combined to obtain a final liquid solution.

5. Heat Exchange

The final liquid solution was subjected to heat exchange to remove water and to obtain a concentrated bean extract.

6. Drying

The concentrated bean extract was then dried by lyophilization to obtain the purified amylase inhibitor. Approximately 120 g of purified amylase inhibitor was obtained from the initial 1000 g of beans.

1. A purified amylase inhibitor obtained by a process comprising the steps of:
(i) grinding white kidney beans to produce coarsely ground beans;
(ii) extracting impurities from the coarsely ground beans by subjecting the beans to supercritical carbon dioxide, under vacuum pressure, to obtain a bean mass;
(iii) incubating the bean mass in deionized water to obtain a first bean suspension containing a first solid component and first liquid component;
(iv) separating out the first solid component from the bean suspension and retaining the first liquid component;
(v) incubating the first solid component in deionized water to obtain a second bean suspension containing a second solid component and a second liquid component;
(vi) separating out the second solid component from the second bean suspension and retaining the second liquid component;
(vii) combining the first liquid component and the second liquid component to obtain a final liquid solution;
(viii) subjecting the final liquid solution to heat exchange to obtain a concentrated bean extract;
(ix) drying the concentrated bean extract;
whereby a purified amylase inhibitor is obtained.

2. The purified amylase inhibitor according to claim 1, wherein the separating of steps (iv) and (vi) of the process is carried out by filtering through a filter press.

3. The purified amylase inhibitor according to claim 1, wherein the separating of steps (iv) and (vi) of the process is carried out by centrifugation.

4. The purified amylase inhibitor according to claim 1, wherein the drying of step (ix) of the process is carried out by spray drying the concentrated bean extract to form a dried bean extract, and wherein the method further comprises the steps of:
(x) rehydrating the dried bean extract to form a rehydrated bean extract; and
(xi) lyophilizing the rehydrated bean extract.

5. The purified amylase inhibitor according to claim 1, wherein the drying of step (ix) of the process is carried out by lyophilization.

6. The purified amylase inhibitor according to claim 1, wherein the extracting of step (ii) of the process is carried out at a temperature of about 120-200° F. for about two hours.

7. The purified amylase inhibitor according to claim 1, wherein the extracting of step (ii) of the process is carried out at a temperature of about 135-160° F. for about two hours.

8. The purified amylase inhibitor according to claim 1, wherein the extracting of step (ii) of the process is carried out at a temperature of about 145° F. for about two hours.

9. The purified amylase inhibitor according to claim 4, wherein about 40-70% of the dried bean extract of step (x) is rehydrated.

10. The purified amylase inhibitor according to claim 4, wherein about 60% of the dried bean extract of step (x) is rehydrated.

11. A method for inducing weight loss in a mammal in need thereof comprising administering to the mammal, an effective amount of a purified amylase inhibitor according to claim 1.

12. The method according to claim 11, wherein the mammal is a human.

13. A method for improving post-prandial glucose tolerance in a mammal in need thereof comprising administering to the mammal, an effective amount of a purified amylase inhibitor according to claim 1.

14. The method according to claim 13, wherein the mammal is a human.
